(19) United States
(12) Patent Application Publication
(10) Pub. No.: US 2005/0239180 A1
(43) Pub. Date: Oct. 27, 2005

(54) Nucleotide sequence coding for a mannitol-2 dehydrogenase and method for the production of d-mannitol

(76) Inventors: Hermann Sahm, Julich (DE); Gerald Hahn, Herzogenrath (DE); Stephanie Bringer-Meyer, Julich (DE); Bjorn Kaup, Montabaur (DE); Claudia Hemmerling, Braunschweig (DE); Martin Walter, Bortfield (DE); Dieter Wullbrandt, Gross Denkte (DE)

Correspondence Address: CANTOR COLBURN, LLP, 55 GRIFFIN ROAD SOUTH, BLOOMFIELD, CT, 06002, US

(21) Appl. No.: 10/514,031

(22) PCT Filed: May 6, 2003
(86) PCT No.: PCT/DE03/01456

§ 371 Date: May 25, 2005

(30) Foreign Application Priority Data
May 8, 2002 (DE) 10220848.4

(51) Int. Cl. C12P 7/18; C12N 9/02; C07H 21/04; C12N 15/74; C12N 1/21

(52) U.S. Cl. 435/158; 435/471; 435/252.3; 435/189; 536/23.2

(57) ABSTRACT

The invention relates to a nucleotide sequence coding for the mannitol 2-dehydrogenase and a method for producing D-mannitol. Previously known biocatalytic methods for producing D-mannitol yield only small production rates due to the small number of specific activities of mannitol 2-dehydrogenases used for transforming D-fructose into D-mannitol. D-mannitol production can be improved by supplying a nucleotide sequence which codes for a mannitol 2-dehydrogenase having a higher specific activity. Mannitol production can be increased in a particular manner by creating a regeneration system for reduction equivalents by introducing and/or strengthening a formiate dehydrogenase in a microorganism.

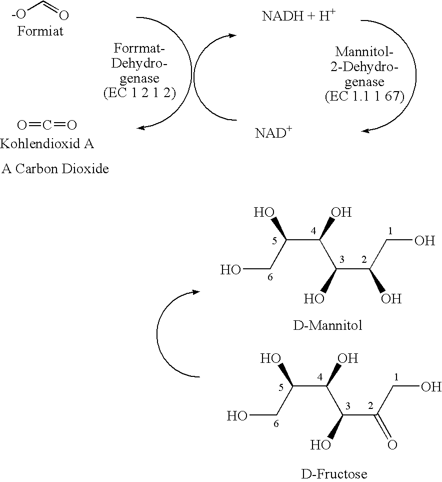

The invention relates to a nucleotide sequence coding for mannitol 2-dehydrogenase and a method for the production of D-mannitol.

The world-wide consumption of the sugar alcohol D-mannitol is 30 000 tonnes per annum. D-mannitol is used in the foodstuffs industry as a sweetener which does not harm the teeth, in medicine as a plasma expander and vasodilator (hexanitro derivative), and in the pharmaceuticals industry in tablet production.

On a commercial scale, D-mannitol has so far been produced by the catalytic hydrogenation, over metal catalysts, of glucose/fructose mixtures from sucrose as the starting materials. Because the catalytic hydrogenation is non-stereospecific, the yield of D-mannitol is only 25-30% with a threefold excess of D-sorbitol (42, 21, 43).

D-mannitol and D-sorbitol differ only in their configuration at the C2 carbon atom. D-mannitol may alternatively be produced by enzymatic hydrogenation of D-fructose in a microbial biotransformation process. Enzymes catalyse their reactions stereospecifically. Slatner et al. (47) describe, for example, an enzymatic process in which a recombinant mannitol dehydrogenase from Pseudomonas fluorescens is isolated and incubated together with a formiate dehydrogenase from Candida boidinii and NAD in a membrane reactor. The use of the formiate dehydrogenase sets up a cycle of NADH reduction and oxidation, NADH being retained in the reaction vessel by the membrane. In this case, it was possible to convert 70-90% of the fructose into D-mannitol. As drawbacks of the method, the authors cite the poor stability of the mannitol dehydrogenase (half life: 50 h; after stabilisation with dithiothreitol: 100 h), and their sensitivity to high temperatures >30° C. and to shearing forces, but a further, major disadvantage is that membrane reactors are unsuitable for large-scale industrial production on account of the high cost of isolated enzymes, and the requisite co-factors and membranes.

Fermentation processes offer a further possible means for D-mannitol production. As early as in 1991, Soetaert et al. (36) obtained yields of 85% in fermentative D-mannitol production using D-fructose/D-glucose mixtures as the substrate. For this, they used the heterofermentative lactic-acid bacterium Leuconostoc pseudomesenteroides ATCC 12291 as the catalysing organism in a fermentation with growing cells; the reduction equivalents necessary for the reduction of fructose to D-mannitol originated from the oxidation of glucose to organic acids (36). Apart from the problem that the substrate fructose is only 85% converted to D-mannitol, D-glucose is an expensive electron donor to use. Furthermore, contamination of the target substance with organic acids during fermentation represents a further disadvantage, as these organic acids have to be removed by means of complex stages of the process. Soltaert et al. proposed electrodialysis for this purpose (36, 37). With fermentation with growing cells, 100% conversion of the substrate to the product can be achieved, since some of the substrate is used in cell construction or in the production of new biomass.

The key enzyme for the enzyme-catalysed reductive reaction of D-fructose to D-mannitol is mannitol-2-dehydrogenase. The literature discloses three mannitol-2-dehydrogenases which, moreover, have been described in respect of their biochemical properties and nucleotide/amino acid sequences. These include the mannitol-2-dehydrogenase from Pseudomonas fluorescens DSM 50106 (4, 34), from Rhodobacter sphaeroides Si4 (32), and from Agaricus bisporus (11, 38). The first two belong to the long-chain dehydrogenase/reductase (LDR) protein family, and the last to the short-chain dehydrogenase/reductase (SDR) protein family. However, the specific activity of these enzymes in the reduction of D-fructose to D-mannitol is only around 40 to 90 U/mg.

The aim of the invention is therefore to provide a system which does not have the aforementioned disadvantages, and to make new measures available for the improved microbial production of D-mannitol.

The designation D-mannitol should be understood as also referring to D-mannit.

Within the scope of this invention, all nucleotide sequences coding for a mannitol-2-dehydrogenase will, in what follows, be grouped together under the name “mdh-gene sequence”; the enzyme mannitol-2-dehydrogenase will be given the common designation “MDH”.

One aim of the invention is to provide a nucleotide sequence coding for an MDH, containing
(i) a nucleotide sequence, represented in SEQ ID No. 1, or
(ii) comprising at least one nucleotide sequence which corresponds to nucleotide sequence i) within the range of degeneracy of the genetic code; or
(iii) comprising at least one nucleotide sequence which hybridises with a nucleotide sequence complementary to nucleotide sequence i) or ii), and optionally
(iiii) comprising functionally-neutral sense mutations in i). Here, the term functionally-neutral sense mutations means the exchange of chemically similar amino acids, e.g. glycine with alanine, or serine with threonine.

The nucleotide sequences according to the invention are characterised in that they are isolated from the Lactobacteriaceae family, preferably from the genus Leuconostoc, especially preferably from Leuconostoc pseudomesenteroides, and quite especially preferably from Leuconostoc pseudomesenteroides ATCC 12291.

In accordance with the Budapest Treaty, the mdh-gene sequence according to the invention was deposited on 20 Feb. 2002 with the DSMZ [German Collection of Microorganisms and Cell Cultures) as plasmid DNA (pQE80Lmdh) under the accession number: DSM 14824.

A nucleotide sequence, a nucleic acid or a nucleic-acid fragment according to the invention is understood as a polymer of RNA or DNA which may be single-stranded or double-stranded and may optionally contain natural, chemically synthesised, modified or artificial nucleotides. The term DNA polymer here includes genomic DNA, cDNA or mixtures thereof.

The sequence regions preceding (5′ or upstream) or following (3′ or downstream) the coding regions (structural genes) are also included according to the invention. Sequence regions having regulatory function are included in particular. These may influence transcription, RNA stability or RNA processing, as well as translation. Examples of regulatory sequences are, inter alia, promoters, enhancers, operators, terminators or translation enhancers.

A further subject of the invention is a gene structure containing at least one of the previously described nucleotide sequences coding for an MDH, as well as regulatory sequences operatively linked thereto which control the expression of the coding sequences in the host cell. Operative linkage is understood to mean the sequential arrangement of, for example, promoter, coding sequence, terminator and optionally other regulatory elements in such a way that each of the regulatory elements is able to fulfil its function in respect of expression of the coding sequence as intended. A promoter inducible by IPTG (isopropyl-β-thiogalactoside) may be cited here by way of example.

Production of a gene structure is realised by fusion of a suitable promoter with at least one nucleotide sequence according to the invention in accordance with currently valid recombination and cloning techniques such as, for example, those described in (24).

The present invention furthermore relates to a vector containing at least one nucleotide sequence of the previously described type coding for an MDH, regulatory neucleotide sequences operatively linked thereto, as well as supplementary nucleotide sequences for the selection of transformed host cells, for replication within the host cell or for integration into the corresponding host-cell genome. The vector according to the invention may furthermore contain a gene structure of the aforementioned type. Suitable vectors are ones which are replicated in the host cells.

By exploiting the nucleotide sequences according to the invention, corresponding probes or primers may be synthesised and used, for example with the aid of the PCR technique, to amplify and isolate analog genes from other microorganisms, for example from the genus Leuconostoc.

The subject of the present invention is thus also a probe for identifying and/or isolating genes coding for proteins involved in the biosynthesis of D-mannitol, this probe being produced on the basis of the nucleotide sequences of the previously described type according to the invention and containing a label suitable for detection. The probe may be a partial portion of the sequence according to the invention, for example from a conserved region, which is able to hybridise specifically with homologous nucleotide sequences under stringent conditions. A great many suitable labels are known from the literature. Guidance in this regard is available to the person skilled in the art, for example, in the following bibliographical references (8, 18, 28).

An aim of the invention is to provide an MDH, coded for by a nucleotide sequence according to the invention, according to SEQ ID No. 1 or variations thereof of the previously described type, having improved fructose-reducing activity by comparison with previously known MDHs. In the present invention, this activity is determined photometrically via the decrease in the NADH concentration for the reduction reaction: D-fructose+NADH+H⁺→D-mannitol+NAD⁺.

The present invention also relates to an MDH having an amino-acid sequence selected from the sequence according to SEQ ID No. 2 or a modified form of this polypeptide sequence or isoform thereof or mixtures thereof.

The polypeptides according to the invention are characterised in that they originate from the Lactobacteriaceae family, preferably from the genus Leuconostoc, especially preferably from Leuconostoc pseudomesenteroides, and quite especially preferably from Leuconostoc pseudomesenteroides ATCC 12291.

Isoforms are understood to be enzymes having identical or comparable substrate specificity and specificity of action, but which have differing primary structures.

Modified forms according to the invention are understood as enzymes in which variations are present in the sequence, for example at the N-end and/or C-end of the polypeptide or in the region of conserved amino acids, without, however, impairing the function of the enzyme. These modifications may be made by known methods in the form of amino-acid substitutions.

A further aim of the invention to provide microorganisms containing, in replicable form, at least one nucleic acid of the previously described type according to the invention, the expression of which nucleic acid is amplified and/or the copy number of which is increased by comparison with the corresponding genetically unmodified microorganism. The present invention similarly includes a genetically modified microorganism containing, in replicable form, a gene structure or a vector of the previously described type. A subject of the present invention is furthermore a genetically modified microorganism containing at least one polypeptide according to the invention with the function of an MDH of the previously described type which has increased activity by comparison with the corresponding genetically unmodified microorganism. The microorganisms according to the invention may originate from the genus Bacillus, Lactobacillus, Leuconostoc, the Enterobacteriaceae or methylotrophic yeasts, fungi and from all microorganisms also used in the foodstuffs industry. The following suitable microorganisms may be cited by way of example: Achromobacter parvolus, Methylobacterium organophilum, Mycobacterium formicum, Pseudomonas spec. 101, Pseudomonas oxalaticus, Moraxella sp., Agrobacterium sp., Paracoccus sp., Ancylobacter aquaticus, Pseudomonas fluorescens, Rhodobacter sphaeroides, Rhodobacter capsulatus, Lactobacillus sp., Lactobacillus brevis, Leuconostoc pseudomesenteroides, Gluconobacter oxydans, Candida boidinii, Candida methylica, or also Hansenula polymorpha, Aspergillus nidulans or Neurospora crassa or Escherichia coli.

The aim is furthermore to produce a method for the production of D-mannitol, with which improved yields and higher productivities may be achieved. This comprises both the insertion of nucleotide sequences according to the invention or of a part of such sequences, which code for an MDH, or of an allele, homologue or derivative thereof, into a host system and the amplification of an MDH-coding nucleotide sequence already present in a microorganism, wherein the gene expression and/or the activity of the correspondingly coded polypeptide is increased by comparison with the corresponding genetically unmodified microorganism, this genetically modified microorganism is used for the microbial production of D-mannitol, and the correspondingly formed D-mannitol is isolated from the culture medium and/or the cells.

The insertion of the nucleotide sequence into a host cell is carried out by methods of genetic engineering. As a preferred method one may cite here the transformation and, especially preferably, the transfer of DNA by electroporation.

To achieve amplification of the mdh-gene sequence or increased gene expression (overexpression), the copy number of the corresponding genes may be increased. Furthermore, the promoter region and/or regulation region and/or the ribosome-binding site, which is located upstream of the structural gene, may be correspondingly modified in such a way that expression occurs at an increased rate. Expression cassettes, which are incorporated upstream of the structural gene, have the same effect. It is also possible to increase expression in the course of microbial D-mannitol production with the use of inducible promoters. Expression is similarly improved by measures to extend the life of mRNA. The genes or gene constructs may either be present in plasmids in differing copy numbers, or be integrated and amplified within the chromosome. Furthermore, the activity of the enzyme itself may also be increased or amplified by preventing the degradation of the enzyme protein. Overexpression of the relevant genes may alternatively also be achieved by modifying the composition of the media and the culturing technique.

Guidance in this regard is available to the person skilled in the art, inter alia, in Martin et al. (25), Guerrero et al. (9), Tsuchiya and Morinaga (41), Eikmanns et al. (7), Schwarzer and Pühler (33), Rheinscheid et al. (27), LaBarre et al. (16), Malumbres et al. (23), Jensen and Hammer (13), Makrides (22) and in known textbooks of genetics and molecular biology.

A host system is understood as microorganisms which are all transformable with foreign DNA. According to the invention, these are understood to include microorganisms in which the nucleotide sequence according to the invention is inserted and/or amplified and accordingly expressed. Microorganisms which already have a nucleotide sequence coding for an MDH or the nucleotide sequence according to the invention, such as e.g. Leuconostoc pseudomesenteroides, are therefore similarly to be understood as a host system. As representatives of a suitable host system into which the nucleotide sequence according to the invention is inserted, one may cite the bacterium Escherichia coli and preferably the strain E. coli JM109 (DE3), which may be cultured under standard conditions.

Depending on the requirements, a complex medium such as e.g. LB medium (24) or even a mineral salt medium (15) are suitable culture media. Following appropriate culturing, the bacterial suspension may be harvested and used for further investigation, for example for transformation or for the isolation of nucleic acids in accordance with currently valid methods.

By analogy, this procedure may also be applied to other bacterial strains. The preferred host systems here are bacteria of the genera to Leuconostoc, Bacillus, Lactobacillus, or Enterobacteriaceae and methylotrophic yeasts. In what follows, a number of preferred microorganisms are listed by way of example: Achromobacter parvolus, Methylobacterium organophilum, Mycobacterium formicum, Pseudomonas spec. 101, Pseudomonas oxalaticus, Moraxella sp., Agrobacterium sp., Paracoccus sp., Ancylobacter aquaticus, or also Pseudomonas fluorescens, Rhodobacter sphaeroides, Rhodobacter capsulatus, Lacto bacillus sp., Lactobacillus brevis, Leuconostoc pseudomesenteroides, Gluconobacter oxydans, or methylotrophic yeasts such as Candida boidinii, Candida methylica, or also Hansenula polymorpha, fungi such as Aspergillus nidulans and Neurospora crassa, as well as all microorganisms also used in the foodstuffs industry. The present invention furthermore includes bacterial strains which are characterised as D-mannitol producing mutants or production strains. These may be produced e.g. on the basis of wild-type strains by conventional (chemical or physical) methods or by methods of genetic engineering.

The genetically modified microorganisms produced according to the invention may be cultured either continuously or discontinuously in batches (batch cultivation), or by the fed-batch method or repeated fed-batch method for the purpose of producing D-mannitol. A brief account of known culturing methods is given in Chmiel's book (5) or in that of Storhas (39).

The culture medium used must by appropriate means satisfy the requirements of the respective strains. Descriptions of culture media for various microorganisms are contained in the Manual of Methods for General Bacteriology (1). Sugars and carbohydrates such as e.g. glucose, sucrose, lactose, fructose, maltose, molasses, starch and cellulose may be used as the carbon source. These substances may be used in isolation or as a mixture. The nitrogen source used may be organic nitrogen-containing compounds such as peptones, yeast extract, meat extract, malt extract, corn steep liquor, soybean flour and urea, or inorganic compounds such as ammonium sulfate, ammonium chloride, ammonium phosphate, ammonium carbonate and ammonia nitrate. The nitrogen sources may be used in isolation or as a mixture. The phosphorus source used may be phosphoric acid, potassium dihydrogen phosphate or dipotassium hydrogen phosphate or the corresponding sodium-containing salts. The culture medium must furthermore contain salts of metals such as magnesium sulfate or iron sulfate, which are necessary for growth.

Finally, essential growth-promoting substances such as amino acids and vitamins must be used in addition to the above-mentioned substances. Moreover, suitable precursors may be added to the culture medium. The aforementioned ingredients may be added to the culture in the form of a single batch, or fed in by some suitable means during culturing. The addition of Zn²⁺ to the medium has proved particularly advantageous within the present invention, as this improves the provision of the cells with the metal ion essential for MDH.

To control the pH of the culture, basic compounds such as sodium hydroxide, potassium hydroxide, ammonia or aqueous ammonia or acidic compounds such as phosphoric acid or sulfuric acid may be used in an appropriate manner. Antifoaming agents such as e.g. fatty acid polyglycol esters may be used to control foaming. To maintain plasmid stability, suitable selectively acting substances e.g. antibiotics may be added to the medium. In order to maintain aerobic conditions, oxygen or oxygen-containing gas mixtures, e.g. air, are introduced into the culture. The culturing temperature is normally 20° C. to 40° C., and preferably 30° C. to 37° C. Culturing is continued until a maximum of D-mannitol has formed. This goal is normally achieved within 12 hours to 50 hours.

Analysis of the D-mannitol concentration may be carried out enzymatically/photometrically by K. Horikoshi's method (12), or by high-pressure liquid chromatography (HPLC), as described by Lindroth et al (19).

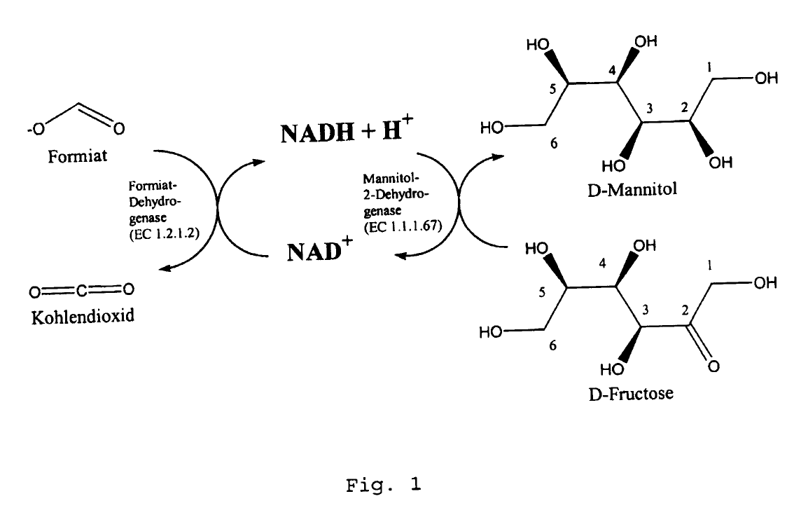

In an advantageous embodiment of the method, a considerable increase in the yield or reaction of the substrate into mannitol is made possible by creating a cofactor-regeneration system. In this embodiment, the substrate is no longer consumed in the preparation of the reduction equivalents for the reduction of fructose to mannitol, rather they are prepared by a second enzyme system. Consequently, more of the substrate is available for conversion to mannitol. One of the most commonly used systems is regeneration with a formiate dehydrogenase, e.g. from Candida boidinii (46). Due to overexpression of this enzyme, together with an arbitrary MDH, preferably from Leuconostoc pseudomesenteroides but also from Rhodobacter sphaeroides, in the microorganism, an oxidation-reduction cycle is set up. Expression of the enzymes may occur within the vector systems suitable for the respective host organism. In the oxidation-reduction cycle thus created, formiate functions as an electron donor and D-fructose as an electron acceptor. Here, the enzyme formiate dehydrogenase catalyses the oxidation of formiate to CO₂ and the enzyme MDH the reduction of D-fructose to D-mannitol (see FIG. 1). The intracellular nicotinic acid amide-adenine-dinucleotide (NAD) pool serves as an electron shuttle between the two enzymes. The oxidation of formiate to CO₂ is thermodynamically advantageous, since the standard free energy of formation ΔGfo for CO₂ is distinctly negative and the CO₂ is removed from the reaction equilibrium by gaseous escape. The increased intracellular NADH concentration resulting from formiate oxidation increases the reductive force for the reduction of D-fructose to D-mannitol, catalysed by MDH. In a further advantageous embodiment of the method, in addition to the carbon sources already mentioned D-glucose is used as a substrate for microbial production. D-glucose may be converted to D-fructose by intracellular conversion with the enzyme Dglucose/xylose isomerase (EC 5.3.1.5) (2). Extracellular conversion is also possible, although intracellular conversion is preferable. The use of D-glucose as a substrate in a microbial method of producing D-mannitol improves the cost-effectiveness of the method, since the D-glucose costs only about one quarter as much as D-fructose.

Microorganisms suitable for the method described include, not only ones into which a formiate dehydrogenase and an MDH are inserted and/or amplified, but also microorganisms which already possess a formiate dehydrogenase, e.g. Achromobacter parvolus, Methylobacterium organophilum, Mycobacterium formicum, Pseudomonas spec. 101, Pseudomonas oxalaticus, Moraxella sp., Agrobacterium sp., Paracoccus sp., Ancylobacter aquaticus, or already possess an MDH. These include microorganisms such as Pseudomonas fluorescens, Rhodobacter sphaeroides, Rhodobacter capsulatus, Lactobacillus sp., Lactobacillus brevis, Gluconobacter oxydans and preferably also Leuconostoc pseudomesenteroides, or microorganisms already possessing both enzymes, the activity of which is amplified in each case. Also suitable are methylotrophic yeasts such as Candida boidinii, Candida methylica, or also Hansenula polymorpha, fungi such as Aspergillus nidulans and Neurospora crassa, as well as all microorganisms also used in the foodstuffs industry.

On the basis of the preamble of claim 1, the aim is achieved according to the invention by the features mentioned in the characterising part of claim 1. The aim is also achieved according to the invention on the basis of the preamble of claim 4, by the features mentioned in the characterising part of claim 4. The aim is furthermore achieved according to the invention on the basis of the preambles of claims 5, 6, 7, 8, 9, 12, 17 and 20, by the features mentioned in the characterising part of claims 5, 6, 7, 8, 9, 12, 17 and 20.

With the nucleic acid and the method according to the invention, improved conversion of the substrate into the product D-mannitol is now possible. Increased productivity is now achieved by comparison with previously known methods, in particular by amplification of the nucleotide sequence according to the invention, as well as a higher yield of D-mannitol. This makes it possible to produce D-mannitol profitably on a large industrial scale. Via creation of the regeneration system with the aid of formiate dehydrogenase, increased conversion of the substrate into the product D-mannitol is made possible for NADH-consuming MDH with resting cells, to an increased degree without the disadvantageous formation of metabolic by-products. Since formiate is far cheaper as an electron donor than glucose, this results in an advantageous cost reduction for the method according to the invention.

Further advantageous developments are mentioned in the dependent claims.

The drawings show, by way of example, results of the method according to the invention as well as a schematic representation of the most important metabolic pathways which play a role in the method.

The figures are as follows:

FIG. 1: Redox cycle with formiate dehydrogenase and MDH;

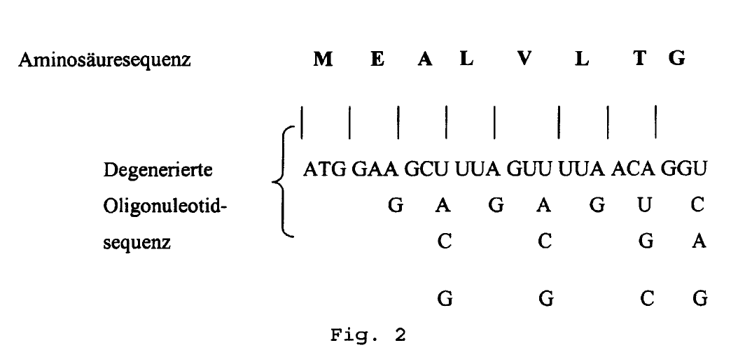

FIG. 2: Derivation of a degenerate 24-base oligonucleotide probe from the N-terminal amino acid sequence of the MDH subunit of Leuconostoc pseudomesenteroides ATCC 12291;

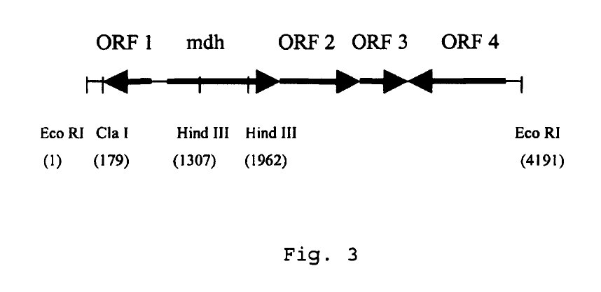

FIG. 3: Gene chart of the 4,191 bp Eco RI fragment isolated from the genomic DNA-plasmid bank of Leuconostoc pseudomesenteroides ATCC 12291 following immunoscreening of the mdh gene. The arrows indicate the direction of translation of the mdh ORF and 4 ORFs.

In what follows the invention will be described with the use of examples.

### EMBODIMENTS

I) Mannitol-2-Dehydrogenase From Leuconostoc pseudomesenteroides ATCC 12291: Purification and Characterisation of the Enzyme; Cloning and Functional Expression of the mdh Gene in Escherichia coli

a) Bacterial Strains and Plasmids

Leuconostoc pseudomesenteroides ATCC 12291 was used as the source for isolation of MDH. E. coli JM 109 (DE 3) (Promega) served as the host organism for production of a plasmid bank for isolation of the genomic DNA from Leuconostoc pseudomesenteroides ATCC 12291. Part of the plasmid bank was produced in pUC18, by ligation of a 4.0-4.5 kb Eco RI fragment of genomic DNA from Leuconostoc pseudomesenteroides ATCC 12291.

b) Culturing Conditions

The following culture medium was used for culturing Leuconostoc pseudomesenteroides ATCC 12291:
Trypton 10 g/l, yeast extract 10 g/l, K₂HPO₄ 10 g/l, D-fructose 20 g/l, D-glucose 10 g/l, vitamin/mineral solution 10 ml/l, in distilled water; pH adjusted to 7.5 with the use of ortho-phosphoric acid.

For subcloning and preparation of plasmid bank of the genomic Leuconostoc DNA, E. coli JM109 (DE 3) was cultured at 170 rpm and 37° C. in Luria-Bertani medium with addition of ampicillin (100 μg/ml) or carbenicillin (50 μg/ml).

c) Determination of the Activity of MDH From Leuconostoc pseudomesenteroides ATCC 12291

In the present invention, the enzyme activity is determined photometrically via the decrease in the NADH concentration for the reduction reaction
D-fructose+NADH+H⁺→D-mannitol+NAD⁺.

The batch for measuring the activity of the MDH contained 200 μM NADH and 200 mM D-fructose in 100 mM potassium phosphate buffer at pH 6.5. The specific activities of the raw extracts and partially purified enzyme isolates have given as units per milligram of protein (U/mg), 1 U being defined as 1 μmol of substrate decrease per minute (20).

d) Determination of the Protein Concentrations

All protein concentration determinations were performed using Bradford's method (3).

e) Separation of Proteins by Polyacrylamide Gel Electrophoresis

Purity analyses of raw extracts and partially purified enzyme isolates, and preparations preparatively on Western blots were carried out by electrophoresis in discontinuous 12% SDS-polyacrylamide gels using Lämmli's method (17).

f) Isolation of Mannitol-2-Dehydrogenase From Leuconostoc pseudomesenteroides ATCC 12291

To isolate the mannitol-2-dehydrogenase, after cellular disintegration familiar to the person skilled in the art (20), the following process steps were carried out: ammonium sulfate precipitation, hydrophobic interaction chromatography (HIC), anion exchange chromatography I (IEC I), anion exchange chromatography II (IEC II), size exclusion chromatography (SEC), and chromatofocusing pH 5-4. These methods are generally known to the person skilled in the art, and may for example be inferred from (20).

At pH=5.35, the specific activity of the MDH for the reduction of D-fructose to D-mannitol was 450 U/mg.

Samples from the purification steps were analysed by SDS-PAGE. A homogenous band was observed at 43 kDa after the last step.

g) Characterisation of the Mannitol-2-Dehydrogenase From Leuconostoc pseudomesenteroides ATCC 12291

The native molecular weight of the MDH was measured as 177 kDa by size exclusion chromatography. The isoelectric point of the enzyme is at pH 4.3-4.4. These measurements were carried out using methods generally known to the person skilled in the art and may, for example, be inferred from Lottspeich and Zorbas (20).

The results for the molecular weight of the native and of the dissociated enzyme lead one to conclude that mannitol-2-dehydrogenase from Leuconostoc pseudomesenteroides ATCC 12291 is a homotetrameric enzyme.

h) Molecular-Genetic Methods

The isolation of genomic DNA from Leuconostoc pseudomesenteroides ATCC 12291, the isolation of DNA fragments from agarose gels, the labelling of DNA probes with digoxigenin-modified dUTP, and immunological detection and DNA-DNA hybridisation (Southern blot) were carried out by methods familiar to the person skilled in the art (24).

The aminoterminal sequencing of the 43 kDa-enzyme subunit by means of Edman degradation and subsequent HPLC analysis yielded the octameric amino-acid sequence MEALVLTG. Using codon usage statistics for Leuconostoc pseudomesenteroides (14), a 2048-fold degenerate oligonucleotide probe for detection of the mannitol-2-dehydrogenase gene in genomic DNA of Leuconostoc pseudomesenteroides ATCC 12291 was derived (see FIG. 2). The 24 bp-DNA probe was provided with a digoxenin-11-dUTP tail at the 3′ end and served for the immunoscreening of partial plasmid banks of genomic DNA from L. pseudomesenteroides ATCC 12291. By this pathway, a 4.2 kb DNA fragment was isolated (FIG. 3). With suitable primers, the mdh gene from this fragment was amplified, ligated into the vector pET24a(+), and transformed and expressed in E. coli BL21 (DE3). Cell extracts from E. coli BL21 (DE3) pET24a (+) Lmdh showed, following induction in SDS-polyacrylamide gel electrophoresis, a pronounced overexpression band at 55.2 kDa and a specific activity of the mannitol-2-dehydrogenase of 102.23 U/mg of protein, whereas the controls (cells without plasmid, cells with blank plasmid) showed no activity.

The nucleotide sequence and the derived amino-acid sequence of the mdh gene from L. pseudomesenteroides ATCC 12291 is shown in sequence ID Nos. 1 and 2 respectively.

II) Biotransformation of D-Fructose to D-Mannitol With a Recombinant E. coli Strain

In a recombinant E. coli strain, the enzymes formiate dehydrogenase (EC 1.2.1.2) and mannitol-2-dehydrogenase (EC 1.1.1.67) were overexpressed in order to establish an oxidation-reduction cycle in the cells. In this oxidation-reduction cycle, hydrogen is transferred from formiate via cellular NAD⁺ to D-fructose, during which D-fructose is reduced to give D-mannitol (see FIG. 1).

(a) Strains and Vectors

Strains E. coli BL21 (DE3) Gold (Stratagene) and E. coli JM109 (DE3) (Promega) were used. pET-28a (+) RspmdhNC (10) coding for the ORF of mannitol-2-dehydrogenase from Rhodobacter sphaeroides Si4 and pBTac2FDH coding for the formiate dehydrogenase from Candida boidinii (35) were used as vectors.

For the biotransformation, chemically competent E. coli BL21 (DE3) Gold was cotransformed with pET28a (+) RspmdhNC and pBTac2FDH and selected on LB-agar plates with 50 μg/ml of carbenicillin and 30 μg/ml of kanamycin. E. coli BL21 (DE3) Gold was furthermore transformed either with pET-28a (+) RspmdhNC or with pBTac2FDH alone. Selection of the transformands was performed on LB-agar plates with either 50 μg/ml of carbenicillin (pBTac2FDH) or 30 μg/ml of kanamycin (pET-28a (+) RspmdhNC). LB-agar plates for E. coli BL21 (DE3) Gold transformed with pET-28a (+) RspmdhNC additionally contained 1% (v/v) D-glucose to prevent the basal expression of mannitol-2-dehydrogenase.

(b) Culturing and Expression

For expression of the enzymes, a single colony of the transformands was pre-cultured with the corresponding antibiotics overnight at 30° C. and with agitation at 170 rpm and re-inoculated into fresh LB medium with 1% (v/v) D-glucose and corresponding antibiotics. Expression of the enzyme was induced with 1 mM IPTG final concentration, and the cultures brought on for a further 5 hours at 27° C. The cell mass from ⅖ of the culture volume was used for enzymatic determinations, and the cell mass from ⅗ of the culture volume was used for the biotransformation. The cells were harvested at 4000 g for 5 min (Beckmann JA-10).

(c) Biotransformation

Following the overexpression of formiate dehydrogenase and MDH in E. coli, non-growing cells were used in a biotransformation. Portions of 2.2 g of induced cells of E. coli BL21 (DE3) Gold pET-28a (+) RspmdhNC/pBTac2FDH were washed with 100 mM potassium phosphate buffer of pH 6.5 and re-suspended in 200 ml of reaction solution with 500 mM of D-fructose and 500 mM of sodium formiate in 100 mM of potassium-phosphate buffer of pH 6.5. The batches were agitated in 300-ml baffled flasks at 100-120 rpm and 30° C. for 48 h. 5-ml samples of the supernatant were withdrawn at times 0, 3, 13, 20, 27, 38, 45 and 48 h after the start of the reaction for measurement of the concentrations of formiate, D-fructose and D-mannitol. The samples were centrifuged at 5000 g for 15 min (Heraeus 3360), the supernatant was 0.2 μm-filtered and stored for HPLC measurement at −20° C. As a control, 5.5 g of non-induced cells of E. coli BL21 (DE3) Gold pET-28a (+) RspmdhNC/pBTac2FDH were used in the biotransformation in the same way.

The concentration determinations of formiate, D-fructose and D-mannitol in the reaction supernatant and in the cell-free raw extract were carried out using an HPLC system (Merck/Hitachi).

Table 1 shows, by way of example, results achieved with transformed microorganisms.

TABLE 1
Production of D-mannitol and consumption of D-fructose and sodium formiate
during a biotransformation

| | | E. coli BL21 (DE3) | E. coli BL21 (DE3) | E. coli BL21 (DE3) |
|---|---|---|---|---|
| | | pET- | pET- | pET- |
| | Substance | 28a (+)RspmdhNC | 28a (+)RspmdhNC | 28a (+)RspmdhNC |
| | production/ | pBTac2FDH | pBTac2FDH | pBTac2FDH |
| | consumption | Induced | Induced | Non-induced |
| | (g/g wet cell mass) | Batch 1 | Batch 2 | control |
| 13 h after | D-mannitol | 0.36 | 0.28 | 0.02 |
| start of | production | | | |
| reaction | D-fructose | 0.97 | 0.91 | 0.01 |
| | consumption | | | |
| | Formiate | 0.33 | 0.22 | 0.04 |
| | consumption | | | |
| 48 h after | D-mannitol | 0.42 | 0.37 | 0.05 |
| start of | production | | | |
| reaction | D-fructose | 1.32 | 1.0 | 0.47 |
| | consumption | | | |
| | Formiate | 0.49 | 0.54 | 0.20 |
| | consumption | | | |

It was demonstrated that the parallel overexpression of formiate dehydrogenase and mannitol-2-dehydrogenase in E. coli leads to a production of the-mannitol by these cells in a reaction medium with D-fructose and formiate.

(d) Enzymatic Determinations

Enzymatic activities of formiate dehydrogenase and MDH in the cell-free extract were measured photometrically at 340 nm. The test batch for the formiate dehydrogenase contained 2 mM NAD+ and 200 mM sodium formiate in 100 mM potassium phosphate buffer at pH 6.5. These high co-enzyme and substrate concentrations were necessary on account of the high Kₘ values of the formiate dehydrogenase, for the purpose of reaching the maximum rate (35). The pH value corresponded to the biotransformation conditions. The batch for measuring the MDH activity was as described in section Ic). Both determinations were carried out at 30° C. After 2 min of measuring the basal activity without the substrate, the enzyme-specific activity following addition of the substrate was measured for a further 2 min. To calculate the activity, the specific absorption coefficient of NAD at 340 nm E=6220 M⁻¹cm⁻¹ was used. A unit was defined as the reduction or oxidation of 1 μmol NAD per minute at pH 6.5 and 30° C.

The specific activity of the formiate dehydrogenase in the cell-free raw extract of E. coli BL21 (DE3) Gold pET-28a (+) RspmdhNC/pBTac2FDH was 0.14 U/mg. Slusarczyk et al. measured the specific activity of the purified formiate dehydrogenase as 6.5 U/mg (35). On the basis of this value, the proportion of formiate dehydrogenase in the soluble cellular protein in the induced E. coli BL21 (DE3) Gold pET-28a (+) RspmdhNC/pBTac2FDH is calculated as 2.2%.

As regards the stability of MDH, no reduction in the specific activity in the cell-free raw extract was detected even 48 h after the start of the reaction (Table 2).

The specific activity of MDH in the cell-free raw extract is found to be constantly high, with a value of 10-12 U/mg. The parallel expression of formiate dehydrogenase in E. coli BL21 (DE3) Gold pET-28a (+) RspmdhNC/pBTac2FDH resulted in no reduction of the specific activity of MDH in the cell-free raw extract.

TABLE 2
Specific enzyme activities of MDH and formiate dehydrogenase

| | E. coli BL21 | E. coli BL21 | E. coli BL21 |
|---|---|---|---|
| | (DE3) pET-28a | (DE3) pET- | (DE3) pET- |
| | (+)RspmdhNC | 28a (+)RspmdhNC | 28a (+)RspmdhNC |
| | pBTac2FDH | pBTac2FDH | pBTac2FDH |
| | Induced | Induced | Non-induced |
| | Batch 1 | Batch 2 | control |
| MDH | 12.08 | 11.07 | 0.13 |
| (U/mg) | | | |
| after | | | |
| induction | | | |
| FDH | 0.14 | 0.14 | 0.04 |
| (U/mg) | | | |
| after | | | |
| induction | | | |
| MDH | 14.97 | 16.46 | 0.17 |
| (U/mg) 48 h | | | |
| after start | | | |
| of reaction | | | |
| FDH | 0 | 0 | 0 |
| (U/mg) 48 h | | | |
| after start | | | |
| of reaction | | | |

The above biotransformation of D-fructose to D-mannitol with a mannitol-2-dehydrogenase from Rhodobacter sphaeroides can also be performed in a comparable way with mannitol-2-dehydrogenase from Leuconostoc pseudomesenteroides. The nucleotide sequence according to the invention may be transformed and expressed in the corresponding host microorganism, e.g. E. coli, by methods known to the person skilled in the art, and this microorganism then used for the microbial production of D-mannitol.

### LITERATURE

1. American Society for Bacteriology: Manual of Methods for General Bacteriology, Washington D.C., USA (1981);
2. Bhosale, S. H., Rao M. B., Deshpande V. V. (1996). Microbiol. Rev. 60: 280-300;
3. Bradford, M. M. (1976): A rapid and sensitive method for the quantification of microgram quantities of protein utilizing the principles of protein-dye binding. Anal. Biochem. 72: 248-254;
4. Brünker P., Altenbuchner J., Mattes R. (1998): Structure and function of the genes involved in mannitol, arabitol and glucitol utilization from Pseudomonas fluorescens DSM 50106. Gene 206: 117-126.
5. Chmiel: Bioprozesstechnik 1. Einführung in die Bioverfahrenstechnik [Bioprocessing techniques 1. Introduction to biochemical engineering]; Gustav Fischer Verlag, Stuttgart (1991);
6. Dubbendorff J. W., Studier F. W. (1991): Controlling basal expression in an inducible T7 expression system by blocking the target T7 promoter with lac repressor. J. Mol. Biol. 219: 45-59;
7. Eikmanns et al. (1991). Gene 102: 93-98;
8. Gait: Oligonucleotide synthesis: a practical approach (IRL Press, Oxford, UK, 1984) and Newton and Graham: PCR (Spektrum Akademischer Verlag, Heidelberg, Germany, 1994);
9. Guerrero et al. (1994): Gene 138, 35-41;
10. Hahn G. (2000): Entwicklung eines mikrobiellen Verfahrens zur Herstellung von D-mannitol [Development of a microbial method for producing D-mannitol]. Thesis, University of Düsseldorf;
11. Horer S., Stoop J., Mooibroeck H., Baumann U., Sassoon J. (2001): The crystallographic structure of mannitol-2-dehydrogenase NADP⁺ binary complex from Agaricus bisporus. J. Biol. Chem. 276: 27555-27561;
12. Horikoshi K. (1963): Meth. Enzym. Analysis, 3^{rd} ed., vol. 6; H. U. Bergmeyer, ed., Verlag Chemie, Weinheim;
13. Jensen and Hammer (1998). Biotechnology and Bioengineering 58, 191-195;
14. Kazusa Research Institute, Chiba, Japan: Codon Usage Database;
15. Keilhauer, C. et al. (1993): J. Bacteriol. 175, 5593-5603;
16. LaBarre et al. (1993): J. Bacteriol. 175, 1001-1007;
17. Lammli U. K. (1970): Cleavage of structural proteins during assembly of the head of bacteriophage T4. Nature 19: 680-685;
18. Liebl et al. International Journal of Systematic Bacteriology (1991) 41: 255-260;
19. Lindroth et al. (1979). Analytical Chemistry 51:1167-1174;
20. Lottspeich F. and Zorbas H., eds. (1998) Bioanalytik [Bioanalysis]. Spektrum Akademischer Verlag GmbH, Heidelberg, Berlin;
21. Makkee M., Kieboom A P G, Van Bekkum H. (1985): Production methods of D-mannitol. Starch/Stärke 37: 136-140;
22. Makrides (1996). Microbiological Reviews 60: 512-53;
23. Malumbres et al. (1993): Gene 134, 15-24;
24. Maniatis T., Fritsch E. F. and Sambrook J.: Molecular Cloning: A Laboratory Manual, Cold Spring Harbor Laboratory, Cold Spring Harbor, N.Y. (1989);
25. Martin et al. (1987): Bio/Technology 5, 137-146;
26. Morton N., Dickerson A. G., Hammond J. B. W. (1985): Mannitol metabolism in Agaricus bisporus: purification and properties in mannitol dehydrogenase. J. Gen. Microbiol. 131: 285-2890;
27. Reinscheid et al. (1994). Applied and Environmental Microbiology 60, 126-132;
28. Roche Diagnostics (Mannheim, Germany): The DIG System Users Guide for Filter Hybridization;
29. Rosenberg A H, Lade B N, Chul D, Lin S, Dunn J J, Studier F W (1987): Vectors for selective expression of cloned DNAs by T7 RNA polymerase. Gene 56: 125-135;
30. Sakai S., Yamanaka K. (1968): Crystalline D-mannitol: NAD oxidoreductase from Leuconostoc pseudomesenteroides. Agric. Biol. Chem. 32: 894-898;
31. Sakai S., Yamanaka K. (1968): Crystalline D-mannitol: NAD⁺ oxidoreductase from Leuconostoc pseudomesenteroides. Biochem. Biophys. Acta 151: 684-686;
32. Schneider K. H., Giffhorn F., Kaplan S. (1993): Cloning, nucleotide sequence and characterization of the mannitol dehydrogenase gene from Rhodobacter sphaeroides. J. Gen. Microbiology 139: 2475-2484;
33. Schwarzer and Pühler (1991): Bio/Technology 9, 84-87;
34. Slatner M., Nidetzky B., Kulbe K. D. (1999): Kinetic studies of catalytic mechanism of mannitol dehydrogenase from Pseudomonas fluorescens. Biochemistry 38: 10489-10498;
35. Slusaczyk H., Felber S., Kula M. R., Pohl, M.: Stabilization of NAD-dependent formiate dehydrogenase from Candida boidinii by site-directed mutagenesis of cystein residues. Eur. J. Biochem. 267: 1280-1289;
36. Soetaert (1991): Synthesis of D-mannitol and L-sorbose by microbial hydrogenation and dehydrogenation of monosaccharides. PhD thesis, University of Ghent;
37. Soetaert W., Buchholz K., Vandamme E J (1995): Production of D-mannitol and Dlactic acid by fermentation with Leuconostoc pseudomesenteroides. Agro-Food Industry High-Tech. 6 (1): 41-44;
38. Stoop J. M., Mooibroeck H. (1998): Cloning and characterization of NADP-mannitol dehydrogenase from the button mushroom Agaricus bisporus, and its expression in response to NaCl stress. Appl. Environ. Microbiol. 64: 4689-4696;
39. Storhas: Bioreaktoren und periphere Einrichtungen [Bioreactors and peripheral devices] Vieweg Verlag, Braunschweig/Wiesbaden (1994);
40. Studier F. W., Rosenberg A. H., Dunn J. J., Dubbendorff J. W. (1990): Use of T7 RNA polymerase to direct expression of cloned genes. Methods Enzymol. 185: 45-59;
41. Tsuchiya and Morinaga (1988): Biotechnology 6,428-430;
42. Wisniak J., Simon R. (1979): Hydrogenation of glucose, fructose and their mixture. Ind. Eng. Chem. Prod. Res. Dev. 18: 50-57;
43. Wright L. W. (1974): Sorbitol and mannitol. Chemtech. January 1974: 42-46;
44. Yamanaka K. (1975): D-mannitol dehydrogenase from Leuconostoc pseudomesenteroides. Meth. Enzymol. 41: 138-142;
45. Yamanaka K., Izawa K., Tenmizu K. (1977): Physicochemical properties of crystalline D-mannitol dehydrogenase of Leuconostoc pseudomesenteroides. Agric. Biol. Chem. 41: 1695-1699;
46. Seelbach K., Riebel B., Hummel W., Kula M-R, Tishkov V., Egorov A., Wandrey C. and Kragl, U. (1996): A novel, efficient regenerating method of NADPH using a new formiate dehydrogenase. Tetrahedron Lett. 37, 1377-1380;
47. Slatner M. et al. (1998). Biotransf. 16: 351-363.

1. A nucleotide sequence coding for an MDH, containing
(i) a nucleotide sequence shown in SEQ ID No. 1;
(ii) at least one nucleotide sequence which corresponds to nucleotide sequence (i) within the range of degeneracy of the genetic code; or
(iii) at least one nucleotide sequence which hybridises with the nucleotide sequence complementary to nucleotide sequence (i) or (ii), and optionally
(iiii) functionally-neutral sense mutations in (i).

2. A nucleotide sequence according to claim 1,
characterised in that
it is isolated from the family of the Lactobacteriaceae.

3. A nucleotide sequence according to claim 1,
characterised in that
it is isolated from Leuconostoc pseudomesenteroides.

4. A plasmid pQE80Lmdh,
deposited with the DSMZ under accession number DSM 14824.

5. A gene structure containing at least one nucleotide sequence according to claim 1, and regulatory sequences operatively linked thereto.

6. A vector containing at least one nucleotide sequence according to claim 1, as well as supplementary nucleotide sequences for selection, for replication in the host cell or for integration into the host-cell genome.

7. A probe for identifying and/or isolating genes coding for an MDH,
characterised in that
it is produced on the basis of a nucleotide sequence according to claim 1 and contains a label suitable for detection.

8. MDH or a part thereof,
coded for by a nucleotide sequence according to claim 1.

9. MDH having an amino-acid sequence derived from the nucleotide sequence according to claim 1, represented in Seq ID No. 2, or a modified form of this polypeptide sequence or isoform thereof or mixtures thereof.

10. A polypeptide according to claim 8,
characterised in that
it originates from the family of the Lactobacteriaceae.

11. A polypeptide according to claim 8,
characterised in that
it originates from Leuconostoc pseudomesenteroides.

12. A microorganism containing, in replicable form, a nucleotide sequence according to claim 1, which is amplified and/or its copy number increased by comparison with the corresponding genetically unmodified microorganism.

13. A microorganism according to claim 12,
characterised in that
it contains a gene structure according to claim 5.

14. A microorganism according to claim 12, containing at least one polypeptide which exhibits increased activity by comparison with the corresponding genetically unmodified microorganism.

15. A microorganism according to claim 12,
characterised in that
it originates from the genus bacillus, Lactobacillus, Leuconostoc, the Enterobacteriaceae or methylotrophic yeasts, fungi and from all microorganisms also used in the foodstuffs industry.

16. A microorganism according to claim 12,
characterised in that
it originates from the group Achromobacter parvolus, Methylobacterium organophilum, Mycobacterium formicum, Pseudomonas spec. 101, Pseudomonas oxalaticus, Moraxella sp., Agrobacterium sp., Paracoccus sp., Ancylobacter aquaticus, Pseudomonas fluorescens, Rhodobacter sphaeroides, Rhodobacter capsulatus, Lactobacillus sp., Lactobacillus brevis, Leuconostoc pseudomesenteroides, Gluconobacter oxydans, Candida boidinii, Candida methylica, or also Hansenula polymorpha, Aspergillus nidulans or Neurospora crassa or Escherichia coli.

17. A method for the microbial production of D-mannitol,
characterised in that
microorganisms are used in which the nucleotide sequence according to claim 1 coding for MDH is inserted and/or amplified.

18. A method according to claim 17,
characterised in that
a microorganism transformed with one or more plasmid vectors is used, which bears a plasmid vector for the nucleotide sequence coding for MDH.

19. A method according to claim 17,
characterised in that
microorganisms transformed with the plasmid vector pQE80Lmdh,
deposited under accession number DSM 14824, are used.

20. A method for the microbial production of D-mannitol,
characterised in that
microorganisms are used in which a nucleotide sequence coding for an MDH and a nucleotide sequence coding for a formiate dehydrogenase is inserted and/or amplified.

21. A method according to claim 20,
characterised in that
microorganisms are used in which a nucleotide sequence for MDH is inserted and/or amplified.

22. A method according to either claim 20 or claim 21,
characterised in that
microorganisms are used in which a nucleotide sequence coding for a formiate dehydrogenase and isolated from Candida boidinii is inserted and/or amplified.

23. A method according to claim 20,
characterised in that
a microorganism transformed with one or more plasmid vectors is used, which bears a plasmid vector for the nucleotide sequence coding for MDH and for formiate dehydrogenase.

24. A method according to claim 17,
characterised in that
microorganisms of the genus Bacillus, Lactobacillus, Leuconostoc, the Enterobacteriaceae or methylotrophic yeasts, fungi and microorganisms used in the foodstuffs industry are used.

25. A method according to claim 17,
characterised in that
microorganisms from the group Achromobacter parvolus, Methylobacterium organophilum, Mycobacterium formicum, Pseudomonas spec. 101, Pseudomonas oxalaticus, Moraxella sp., Agrobacterium sp., Paracoccus sp., Ancylobacter aquaticus, Pseudomonas fluorescens, Rhodobacter sphaeroides, Rhodobacter capsulatus, Lactobacillus sp., Lactobacillus brevis, Leuconostoc pseudomesenteroides, Gluconobacter oxydans, Candida boidinii, Candida methylica, or also Hansenula polymorpha, Aspergillus nidulans or Neurospora crassa or Escherichia coli are used.

26. A method according to claim 17,
characterised in that
fructose or glucose is used as the carbon source.

27. A method according to claim 17,
characterised in that
the following steps are carried out:
a) microbial production of D-mannitol with the use of microorganisms in which the nucleotide sequence coding for an MDH and a nucleotide sequence coding for a formiate dehydrogenase is inserted and/or amplified;
b) enrichment of the D-mannitol in the medium or in the cells of the microorganisms, and
c) isolation of the D-mannitol.

28. A method according to claim 27,
characterised in that
microorganisms are used in which the nucleotide sequence coding for MDH is inserted and/or amplified.
